(19) United States
(12) Patent Application Publication
(10) Pub. No.: US 2004/0120937 A1
(43) Pub. Date: Jun. 24, 2004

(54) Blood therapy process

(75) Inventor: James Brian Wilson, Oklahoma City, OK (US)

Correspondence Address: James B. Wilson, P.O. Box 891500, Oklahoma City, OK, 73189, US

(73) Assignee: Therapy Patent Corporation, Oklahoma City, OK

(21) Appl. No.: 10/324,518

(22) Filed: Dec. 19, 2002

(51) Int. Cl. A61K 45/00

(52) U.S. Cl. 424/93.71

(57) ABSTRACT

The new ideal is the transfusion of natural blood, artificial blood plasma or artificial blood plasma mixed with natural blood, while draining natural blood for therapy of poison, snake bite, leukemia, aged persons, other cancers, allergies and other undiscovered uses. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

### BACKGROUND OF THE INVENTION

1. Field of the Invention

The invention is a process for treating health conditions through a blood therapy such as the transfusion and replacement of blood with undesirable characteristics with blood having more optimum qualities. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

2. Description of the Prior Art

The art of blood transfusion for medical benefits is well known. Blood is transfused, that is drained into the patient in order to replace lost blood.

### SUMMARY OF THE INVENTION

The new ideal is the transfusion of natural blood, artificial blood plasma or artificial blood plasma mixed with natural blood, while draining natural blood in poison and snake bite patients. Some poisons and snake bites tend to thicken the blood and use of these procedures may thin the patients' blood. As artificial blood is thinner than natural blood and healthy natural blood is thinner than many poisoned bloods. Also, use of these procedures may help decrease the concentration of poisons in the blood. As some of the poisons in the blood are drained as the blood is drained. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

The new ideal is the transfusion of natural blood, artificial blood plasma or artificial blood plasma mixed with natural blood, while draining natural blood in leukemia patients. Perhaps trying a pure artificial blood plasma environment in the patient, then transfusing natural healthy blood may be tried, perhaps in combination with chemotherapy, antibiotics and antiviral drugs. Leukemia is a cancer of the blood and perhaps changing the diseased blood with healthy blood may have a therapeutic value. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

The new ideal is that transfusion of blood from younger persons to older persons, while at the same time draining older blood from the patient to increase the volume of younger blood in the patient. Many older persons complain of cold feet and other extremities. The blood of young persons may have a greater ability to carry blood gases, nutrients and may circulate better than the blood of older persons. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

### DETAILED DESCRIPTION

### Blood Therapy for Poison and Snake Bite

The new ideal is the transfusion of natural blood, artificial blood plasma or artificial blood plasma mixed with natural blood, while draining natural blood in poison and snake bite patients. Some poisons and snake bites tend to thicken the blood and use of these procedures may thin the patients' blood. As artificial blood is thinner than natural blood and healthy natural blood is thinner than many poisoned bloods. Also, use of these procedures may help decrease the concentration of poisons in the blood. As some of the poisons in the blood are drained as the blood is drained. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

### Blood Therapy for Leukemia

The new ideal is the transfusion of natural blood, artificial blood plasma or artificial blood plasma mixed with natural blood, while draining natural blood in leukemia patients. Perhaps trying a pure artificial blood plasma environment in the patient, then transfusing natural healthy blood may be tried, perhaps in combination with chemotherapy, antibiotics and antiviral drugs. Leukemia is a cancer of the blood and perhaps changing the diseased blood with healthy blood may have a therapeutic value. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

### Blood Therapy for the Aged

The new ideal is that transfusion of blood from younger persons to older persons, while at the same time draining older blood from the patient to increase the volume of younger blood in the patient. Many older persons complain of cold feet and other extremities. The blood of young persons may have a greater ability to carry blood gases, nutrients and may circulate better than the blood of older persons. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

### Further Blood Therapy

The new ideal is the transfusion of natural blood, artificial blood plasma or artificial blood plasma mixed with natural blood, while draining natural blood. Such therapy may have therapeutic value for other cancers, allergies and other undiscovered use. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

I claim:

1. BLOOD THERAPY FOR POISON AND SNAKE BITE
The new ideal is the transfusion of natural blood, artificial blood plasma or artificial blood plasma mixed with natural blood, while draining natural blood in poison and snake bite patients. Some poisons and snake bites tend to thicken the blood and use of these procedures may thin the patients' blood. As artificial blood is thinner than natural blood and healthy natural blood is thinner than many poisoned bloods. Also, use of these procedures may help decrease the concentration of poisons in the blood. As some of the poisons in the blood are drained as the blood is drained. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

2. BLOOD THERAPY FOR LEUKEMIA
The new ideal is the transfusion of natural blood, artificial blood plasma or artificial blood plasma mixed with natural blood, while draining natural blood in leukemia patients. Perhaps trying a pure artificial blood plasma environment in the patient, then transfusing natural healthy blood may be tried, perhaps in combination with chemotherapy, antibiotics and antiviral drugs. Leukemia is a cancer of the blood and perhaps changing the diseased blood with healthy blood may have a therapeutic value. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

3. BLOOD THERAPY FOR THE AGED
The new ideal is that transfusion of blood from younger persons to older persons, while at the same time draining older blood from the patient to increase the volume of younger blood in the patient. Many older persons complain of cold feet and other extremities. The blood of young persons may have a greater ability to carry blood gases, nutrients and may circulate better than the blood of older persons. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.

4. FURTHER BLOOD THERAPY
The new ideal is the transfusion of natural blood, artificial blood plasma or artificial blood plasma mixed with natural blood, while draining natural blood. Such therapy may have therapeutic value for other cancers, allergies and other undiscovered use. Some concerns in such blood therapy, is the blood be live, well warmed and well oxygenated, possibly with a heart lung machine. Also, that blood pressure is carefully monitored. If artificial blood plasma is substituted for whole blood, the concern would be careful monitoring for internal and external bleeding as artificial blood plasma does not clot. These therapies are not warranted to improve health. These are untested medical therapies and the inventor and Therapy Patent Corporation accepts no risk or liability for loss of health or death that may occur do to experimental work that may occur with these therapies. Perhaps testing and experimentation on animals should be considered before testing and experimentation on humans is considered.
